(19) United States
(12) Patent Application Publication
(10) Pub. No.: US 2024/0338800 A1
(43) Pub. Date: Oct. 10, 2024

(54) FEATURE-AWARE DEEP-LEARNING-BASED SMART HARMONIC IMAGING FOR ULTRASOUND

(71) Applicant: CANON MEDICAL SYSTEMS CORPORATION, Tochigi (JP)

(72) Inventors: Ting XIA, Vernon Hills, IL (US); Jian ZHOU, Vernon Hills, IL (US); Liang CAI, Vernon Hills, IL (US); Zhou YU, Vernon Hills, IL (US); Tomohisa IMAMURA, Otawara Tochigi (JP); Ryosuke IWASAKI, Otawara Tochigi (JP); Hiroki TAKAHASHI, Nasushiobara Tochigi (JP)

(73) Assignee: CANON MEDICAL SYSTEMS CORPORATION, Tochigi (JP)

(21) Appl. No.: 18/296,840

(22) Filed: Apr. 6, 2023

(51) Int. Cl. G06T 5/00; G06T 7/00; G06T 7/30

(52) U.S. Cl. CPC G06T 5/70; G06T 7/0012; G06T 7/30; G06T 2207/10132; G06T 2207/20081; G06T 2207/20084; G06T 2207/20221; G06T 2207/30004

(57) ABSTRACT

An apparatus, method, and computer-readable medium having processing circuitry to receive first ultrasound data including at least one harmonic component, and apply the first ultrasound data to inputs of a trained deep neural network model that outputs enhanced ultrasound image data, the deep neural network model having been trained with training data including input ultrasound data and corresponding target ultrasound data having predetermined target features, and output the enhanced ultrasound image data.

### BACKGROUND

### Technical Field

The present disclosure is directed to a deep-learning based framework that takes ultrasound data of various harmonic combinations to generate a desired image with improved resolution, fewer artifacts, improved contrast, as well as deep penetration. The framework can be trained to be feature-aware to generate a desired image with enhanced depth-dependent resolution, as well as customized for patient specific information, including body mass index and demographic information. The framework can split out different harmonics from an ultrasound image.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Ultrasound Tissue Harmonic Imaging (UTHI) is a signal processing technique in which an ultrasound beam insonates body tissues and generates harmonic waves from nonlinear distortion during a transmit phase of a pulse-echo cycle. Tissue harmonic images are obtained by transmitting a frequency spectrum by a transducer and receiving a frequency spectrum that includes fundamental echo in a band of fundamental frequencies (referred to herein as a fundamental frequency) as well as harmonics that originate in the body. In harmonic imaging, the harmonic images are obtained by collecting harmonic signals that are tissue generated, and filtering out the fundamental echo signals, resulting in sharper images. The harmonic signals (harmonics) are multiples of the fundamental frequency. Thus, transmitting a band of frequencies centered at a frequency f will result in the production of harmonic frequency bands centered at 2f, 3f, 4f, etc. (referred to as second-order harmonics, third-order harmonics, fourth-order harmonics, and so on for higher-order harmonics).

Various processes have been utilized to create harmonic signals. One process is to filter out the fundamental echo from the received frequency spectrum (bandwidth receive filtering). A second process is to transmit simultaneous pulses with 180 degrees phase difference (pulse inversion). A third process is to transmit two pulses with opposite phase and with adjacent lines of sight (side-by-side phase cancellation). A fourth process has been to encode a digital signature in a sequence of transmit pulses, and then eliminate the tagged echoes with signal processing on reception (pulse-coded harmonics).

In particular, the pulse-encoding process transmits relatively complex pulse sequences into the body with a unique and recognizable code imprinted on each pulse. The unique code is then recognized in the echoes. Because the fundamental echoes have a specific code, they can be identified and canceled. The remaining harmonic echo is then processed to form the image. This process is especially useful in the near field, because longer encoded pulses produce harmonics more efficiently in the near field than do conventional THI pulses.

Another process is differential tissue harmonic imaging (DTHI). In DTHI, two pulses are transmitted simultaneously at different frequencies, referred to as f₁ and f₂. In addition to their second harmonic frequencies (2f₁ and 2f₂), among others, the sum and the difference of the transmitted frequencies (f₂+f₁ and f₂−f₁, respectively) are generated within the tissue. The second harmonic signal of the lower frequency (2f₁), and the difference frequency (f₂−f₁), are detected by the transducer. Other generated frequency components do not fall within the bandwidth of the transducer. By using DTHI, higher resolution, better penetration, and fewer artifacts can be achieved.

Thus, in ultrasound harmonic imaging, higher-order harmonics can provide images with significantly reduced artifacts, improved contrast-to-noise ratio, and improved lateral resolution. However, the high frequency signal of higher-order harmonics tends to attenuate more in a deep region. FIG. 1A illustrates an example of conventional ultrasound that shows some detail in the far-field region 102. FIG. 1B illustrates an example of third-order harmonics, in which the same region 102 is lacking detail. Thus, the far-field image typically suffers more signal loss. Subsequently, the far-field image generally is obtained using lower-order harmonic imaging.

Accordingly, it is one object of the present disclosure to provide methods and systems for obtaining ultrasound harmonic images with deep penetration, improved signal-to-noise ratio in the far field, as well as improved resolution and reduced artifacts in the near-field.

### SUMMARY

An aspect is an apparatus, that can include processing circuitry configured to receive first ultrasound data including at least one harmonic component; apply the first ultrasound data to inputs of a trained deep neural network model that outputs enhanced ultrasound image data, the deep neural network model having been trained with training data including input ultrasound data and corresponding target ultrasound data having predetermined target features; and output the enhanced ultrasound image data.

A further aspect is a method, that can include receiving first ultrasound data including at least one harmonic component; applying the first ultrasound data to inputs of a trained deep neural network model that outputs enhanced ultrasound image data, the deep neural network model having been trained with training data including input ultrasound data and corresponding target ultrasound data having predetermined target features; and outputting the enhanced ultrasound image data.

A further aspect is a non-transitory computer-readable medium storing a program that, when executed by processing circuitry, causes the processing circuitry to perform a method, including receiving first ultrasound data including at least one harmonic component; applying the first ultrasound data to inputs of a trained deep neural network model that outputs enhanced ultrasound image data, the deep neural network model having been trained with training data including input ultrasound data and corresponding target ultrasound data having predetermined target features; and outputting the enhanced ultrasound image data.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates a conventional ultrasound image, FIG. 1B illustrates a third order harmonics image for the same depth as in FIG. 1A;

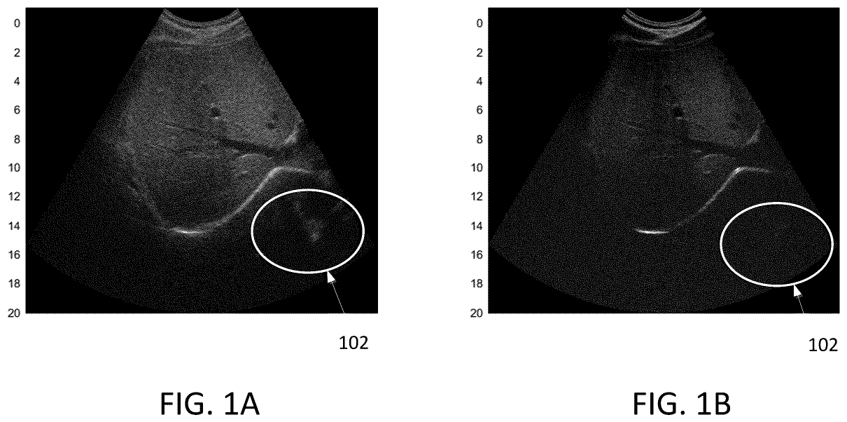

FIG. 2 is a system diagram for an ultrasound imaging system;

FIG. 3 illustrates a conventional method of ultrasound tissue harmonic imaging;

FIG. 4 illustrates a solution in ultrasound tissue harmonic imaging, in accordance with exemplary aspects of the disclosure;

FIG. 5 is an architecture for a residual network, in accordance with an exemplary aspect of the disclosure;

FIG. 6 is a block diagram of a deep neural network for generating desired enhanced harmonics IQ data, in accordance with an exemplary aspect of the disclosure;

FIG. 7 is a block diagram of a deep neural network for generating desired enhanced harmonics B-mode IQ data or ultrasonic image, in accordance with an exemplary aspect of the disclosure;

FIG. 8 illustrates example ultrasound images that are generated by the deep neural network based on an input of IQ1 (fundamental frequency and third-order harmonics) and IQ2 (second-order harmonics);

FIG. 9 illustrates another set of ultrasound images;

FIG. 10 is a block diagram of a deep neural network that is trained directly on a second-order harmonics image and a third-order harmonics image, in accordance with an exemplary aspect of the disclosure;

FIG. 11 is a block diagram of an alternative deep neural network that is trained to extract third-order harmonics and a fusion network model that is trained to fuse the extracted third-order harmonics with second-order harmonics, in accordance with an exemplary aspect of the disclosure;

FIG. 12 is a block diagram of an alternative deep neural network that is trained directly on an IQ1 data/image and second-order harmonics, in accordance with an exemplary aspect of the disclosure;

FIG. 13 is a block diagram of an alternative fusion network that is trained based on extracted third-order harmonics and cleaned second-order harmonics, in accordance with an exemplary aspect of the disclosure;

FIG. 14 is a block diagram of an extraction network that is trained to split second-order harmonics and third-order harmonics from IQ0 data/image, in accordance with an exemplary aspect of the disclosure; and

FIG. 15 is a block diagram illustrating an example computer system 204 for implementing the machine learning training and inference methods according to an exemplary aspect of the disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

High-order harmonic imaging can provide images with fewer artifacts and improved resolution. However, high-order harmonic imaging (higher than second-order harmonics) has limited penetration depth due to the fast attenuation of high frequency signals. Second-order harmonics can penetrate to a greater depth compared to higher-order harmonics. However, second-order harmonic imaging suffers from more artifacts, and reduced contrast-to-noise ratio in the near field. In the case of anatomic structures such as blood vessels, it is desired to obtain a high-order harmonic image having improved contrast.

In conventional ultrasound imaging, to obtain high-order harmonics, the ultrasound transducer needs to send multiple pulses with opposite/desired phases sequentially into a tissue along the same line. Subsequently, the time duration of image acquisition is long, resulting in a low frame rate. However, a high frame rate is particularly important in cases where tissue movement occurs, as the movement can greatly impact the image quality. Also, the conventional ultrasound imaging for high order harmonics is generally limited to the signals received by the transducer. Filters may be used to filter out some frequency bands in order to focus on a desired frequency. However, filtering alone does not enhance the quality of the ultrasound images.

Aspects of this disclosure are directed to a system, device, and method for a deep convolutional neural network to generate enhanced harmonic imaging for ultrasound images. The disclosed method provides harmonics with fewer artifacts, improved contrast, deep penetration as well as substantially increased frame rate using the signals received by the ultrasound transducer. The disclosed method can provide feature-aware, depth-dependent harmonic images, which are not necessarily pure harmonics or pure combined harmonics, but harmonics that are enhanced for improved image quality (being trained to be feature-aware, depth dependent, denoised). The input image will not necessarily contain fundamental frequency data. The disclosed method utilizes different information under different data acquisition modes (including IQ0, IQ1, IQ2, IQ3, as further defined below). With the disclosed deep learning framework, the trained deep neural network (DNN) is robust and can be applied to different ultrasound scan conditions. Unlike conventional approaches, which provide only harmonics or combined harmonics, the disclosed method uses a feature-aware training strategy, and can be customized to different patients with various BMI and/or demographic information, e.g., obese/thin patients, patients with different fat, etc.

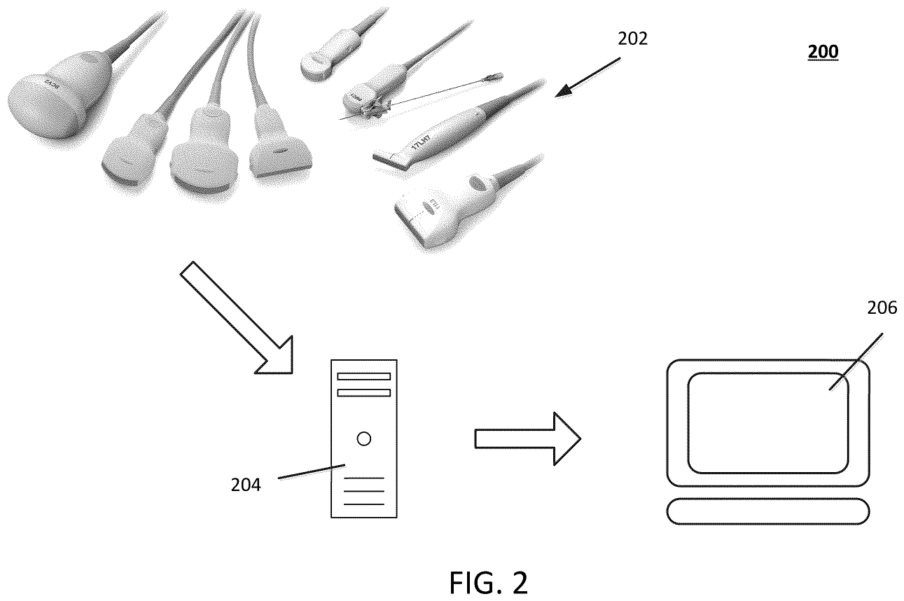

FIG. 2 is a system diagram for an ultrasound imaging system. An ultrasound imaging system 200 can include any of a range of transducers 202. Types of transducers include convex, linear, and sector, as well as those designed for a special purpose. The signals received by a transducer 202 are processed in a computer system 204. The ultrasound imaging system 200 may also include multi-harmonic compounding in which signals from individual beams are merged with overlapping data from adjacent beams.

Ultrasound images are created from sound waves at frequencies above the range audible to humans, typically on the order of 1-10 MHz or above. A transducer 202 emits high frequency waves and records the reflected waves (fundamental frequency), bounced back from interfaces in the tissue, as a series of time-domain signals. One type of ultrasound image is a brightness image, also known as a B-mode image, which is a grayscale, intensity-based representation of the object.

The raw signals that the transducer 202 receives are in the radiofrequency range and are known as radiofrequency (RF) data. A series of signal processing steps are performed in the computer system 204 to convert from the RF data to the ultrasound image, such as a B-mode image. One preprocessing step is to demodulate the RF data to baseband and decimate the signal to reduce the bandwidth required to store the data. This new signal is referred to as an in-phase and quadrature phase (IQ) signal, and is typically represented with complex numbers. In this disclosure, the term IQ and RF data are used interchangeably since they both represent the raw data from the transducer, but in different formats. In addition, embodiments of the deep neural networks of the present disclosure are configured to take as input either the IQ data or an ultrasound image that is based on the IQ data.

The computer system 204 can be embedded in a portable ultrasound machine, can be a remote server, or can be a cloud service that is accessed through the Internet. The ultrasound imaging system 200 includes at least one display device 206 for displaying one or more ultrasound images. The display device 206 can be any of an LCD display, an LED display, an Organic LED display, etc. The display size and resolution are sufficient to display the ultrasound images that are output by the computer system 204.

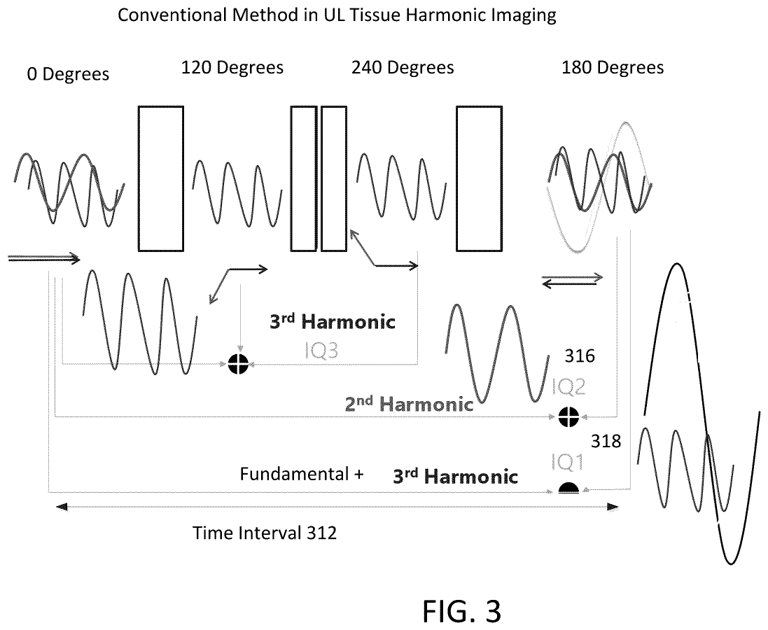

FIG. 3 illustrates a method of ultrasound tissue harmonic imaging. In order to get more signal in the ultrasound far field image, an image can be obtained for both second-order harmonics and third-order harmonics. As described above, one method is the DTHI method in which two pulses are transmitted simultaneously at different frequencies, referred to as f₁ and f₂. In addition to their second harmonic frequencies (2f₁ and 2f₂), among others, the sum and the difference of the transmitted frequencies (f₂+f₁ and f₂−f₁, respectively) are generated within the tissue. The second harmonic signals 316 and 318 of the lower frequency (2f₁), and the difference frequency (f₂−f₁), respectively, are detected by the transducer. However, the method involves a long time interval 312 for pulse generation. Subsequently, the frame rate is slow, leading to low-quality imaging, especially in cases of tissue motion.

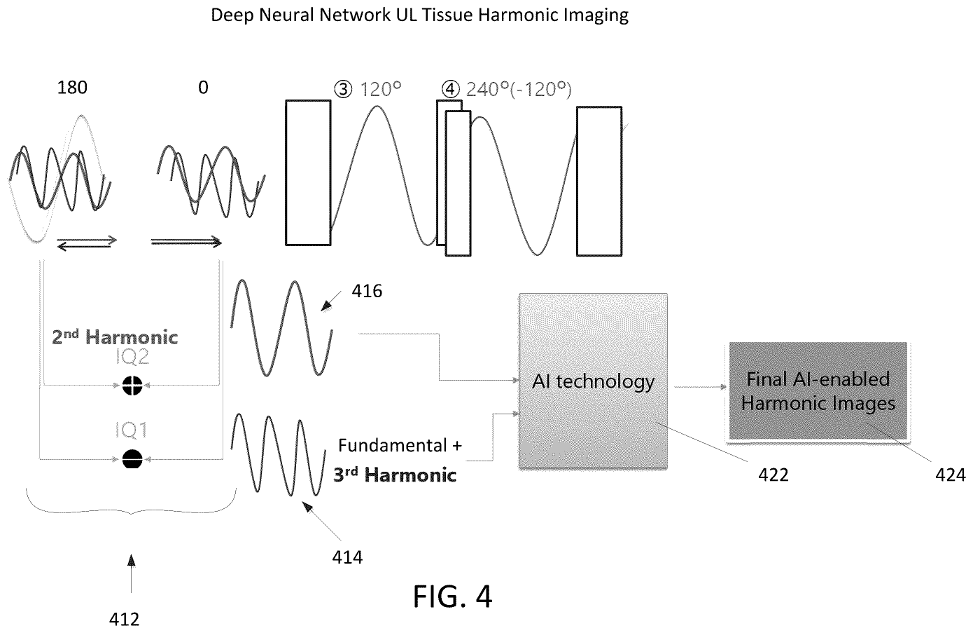

FIG. 4 illustrates a disclosed solution in ultrasound tissue harmonic imaging. In one embodiment, a solution involves pulse generation over a short time interval 412 by obtaining a signal that is a combination of fundamental ultrasound frequency and the third order harmonic 414, and simultaneously obtaining the second-order harmonic 416. A deep neural network 422 generates an estimated third-order harmonic from the combination signal. A deep neural network 422 is trained to effectively generate a denoised second-order harmonic from the obtained second-order harmonic. In addition, a deep neural network 422 is trained to effectively fuse the estimated third-order harmonic and the denoised second-order harmonic to generate an organ-specific fused image.

The short time interval 412 for pulse generation enables a reduction in the effects of motion and a reduction in artifacts. The deep neural network 422 enables improved image quality in a shorter signal acquisition time.

In one embodiment, a deep learning based framework is configured to input IQ data of various different combinations, as received by the transducer 202, and subject to data processing steps in computer system 204, and to output a desired image with improved image quality, fewer near-field artifacts, improved contrast and deeper penetration. The deep learning based framework can directly use second- or third-order harmonics, or alternatively, use data containing fundamental frequencies. The input IQ data can include a combination of a fundamental frequency signal, second-order harmonics signal, and third-order harmonics signal (IQ0). The IQ data can include a combination of fundamental ultrasound frequency and third-order harmonics (IQ1). The input IQ data can also include just a second-order harmonics signal (IQ2), or just a third-order harmonics signal (IQ3). The input IQ data can also be other higher-order harmonics greater than third order.

The deep learning based framework undergoes a training method for a particular target including desired harmonic data or a desired type of image. The framework can learn feature-awareness, depth-dependency, and be customized by considering patients with different body mass index (BMI) and/or demographic information. For example, the trained deep learning based framework is trained to output harmonic IQ data, or a depth-dependent fusion map.

The deep learning based framework can be configured as a structure including multilayer perceptron, convolutional neural network such as U-net, or a fusion network. Convolutional neural networks (CNN) have been used in visual recognition tasks. CNNs can be trained with a large training set of images, for example, 1 million training images as in the ImageNet dataset. ImageNet has 8 layers and millions of parameters. Very deep convolutional networks can be used for large-scale image recognition.

One deep learning network architecture (U-Net) can be trained with far fewer images. Training with thousands of images is usually beyond the reach of typical biomedical tasks. The architecture of U-Net has an upsampling part and a large number of feature channels, which allows the network to propagate context information to higher resolution layers. Subsequently, the original U-Net architecture consists of a contracting path and an expansive path, in which an expansive path is more or less symmetric to a contracting path, giving the U-shaped architecture.

The contracting path follows the architecture of typical a convolutional neural network. It consists of a repeated application of two 3×3 convolutions (unpadded convolutions), each followed by a rectified linear unit (ReLU) and a 2×2 max pooling operation with stride 2 for downsampling. At each downsampling step, the number of feature channels is doubled.

Every step in the expansive path consists of an upsampling of the feature map followed by a 2×2 convolution (“up-convolution”) that halves the number of feature channels, a concatenation with the correspondingly cropped feature map from the contracting path, and two 3×3 convolutions, each followed by a ReLU. At the final layer a 1×1 convolution is used to map each 64-component feature vector to the desired number of classes.

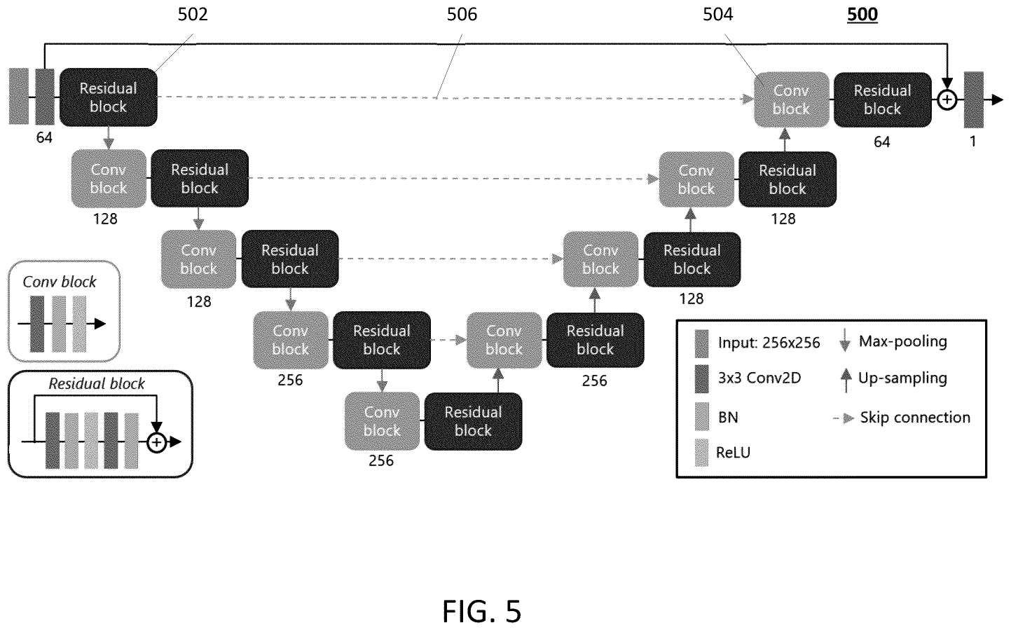

A convolutional neural network that is similar to the U-Net is a residual network (ResNet). The residual network can accommodate a greater depth than the U-Net. FIG. 5 is an architecture for a residual network, in accordance with an exemplary aspect of the disclosure. Embodiments of the deep neural network may be a residual network 500 as in FIG. 5. The residual network 500 can accommodate a greater depth by including skip connections 506. A residual block 502 is built by one convolutional layer (Conv2D, 3×3 kernel size) followed by a batch normalization layer (BN) and an activation layer rectified linear unit (ReLU) and the other convolutional layer followed by a BN. A convolution block 504 is built by one convolution layer.

An alternative to a convolutional neural network for vision is a transformer network such as ViT (Vision Transformer). Vision transformers typically include an image encoder layer. Also, hybrid convolutional-transformer networks have been developed for vision tasks. Embodiments of the deep learning based framework can be configured as a vision transformer.

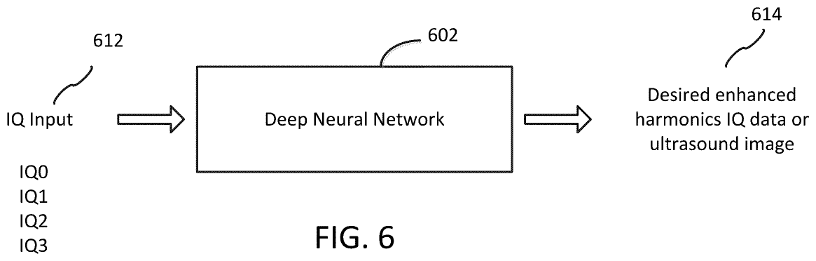

FIG. 6 is a block diagram of a deep neural network for generating desired enhanced harmonics IQ data, in accordance with an exemplary aspect of the disclosure. As used herein, the term desired enhanced harmonics IQ data, as well as desired enhanced harmonics image, is an ultrasound image having better resolution than any of the input harmonics IQ data or input harmonics images, fewer near-field artifacts that the input data or images, better contrast than the input data or images, as well as deep penetration. A desired enhanced harmonic data or image can be used as a training target that is one or more of feature-aware, depth-dependent, and customized based on patients with different body mass index and/or demographic information.

As will be described later, a hardware configuration of the deep neural network can be implemented in a computer system having a processing chip that contains a special purpose machine learning processing unit, or at least a multi-core central processing unit. The software for implementing the deep neural network can include a machine learning framework such as PyTorch, Tensorflow, Keras, to name a few, or using machine learning libraries in programming languages such as Python, C#, and others.

The deep neural network 602 may be any deep neural network. As mentioned above, convolutional neural networks have demonstrated superior results, and in particular the U-Net architecture enables training with a smaller dataset than a full convolutional neural network. Also, other types of deep neural networks, including a multilayered perceptron or a vision transformer can be used to implement the deep neural network 602. Embodiments of the convolutional neural networks for generating ultrasound images are trained to be feature-aware, and can be trained to be depth dependent, and even customized to a class of patients, such as those classified according to body mass index and/or demographic information. The convolutional neural network is made feature-aware through training that is guided towards specific features, e.g., the focus is on local feature refinement during training. Desired feature-aware images are images having reduced artifacts, high contrast, and good lateral resolution. In this disclosure, lateral resolution of ultrasound images are those that have been enhanced by using a narrow ultrasonic beam.

Deep convolutional networks can be used to generate very high resolution images, but to do so requires a very large training dataset, on the order of one million or greater images. In some embodiments, the U-Net architecture or residual network architecture is used to simplify training using a much smaller training set.

As will be described in later examples, the input 612 to the deep neural network 602 can be one or more of higher-order harmonic data, or IQ data containing fundamental frequencies.

The output is a desired enhanced harmonic IQ data or an ultrasound image. The enhanced harmonic IQ data is not necessarily a pure third-order harmonics image (or data), but can include an image having high resolution at a specific penetration depth with few near-field artifacts. The enhanced harmonic IQ data can be a high resolution image for a specific depth of penetration.

Thus, the deep neural network 602 can take as input an image obtained from the fundamental frequency signal and the third-order harmonics component and output a high resolution image for a specific deep penetration depth. The deep neural network 602 can be trained to generate a second-order or a third-order harmonic image with reduced noise. The deep neural network 602 can be trained to generate an ultrasound image that is specific for a patient body mass index and/or a specific demographic.

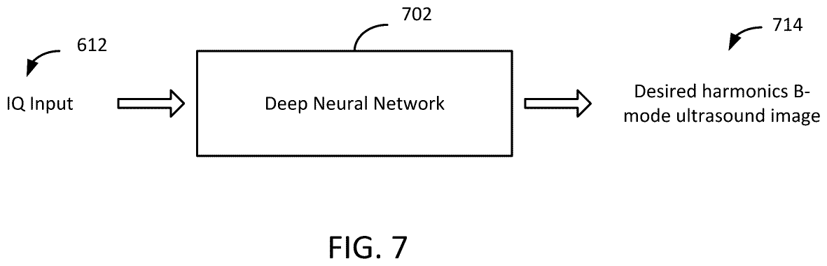

FIG. 7 is a block diagram of a deep neural network for generating desired enhanced harmonics B-mode ultrasonic image, in accordance with an exemplary aspect of the disclosure. B-mode ultrasound uses linear array transducers to simultaneously scan a plane through the body. These echoes are converted by the ultrasound imaging device into a 2D image. As mentioned above, the B-mode ultrasound image is a brightness image, which is a grayscale, intensity-based representation of the object.

The deep neural network 702 can be trained to generate a harmonics B-mode image 714 with reduced noise. The deep neural network 702 can generate a harmonics B-mode ultrasound image 714 that is specific for a patient body mass index and/or a specific demographic.

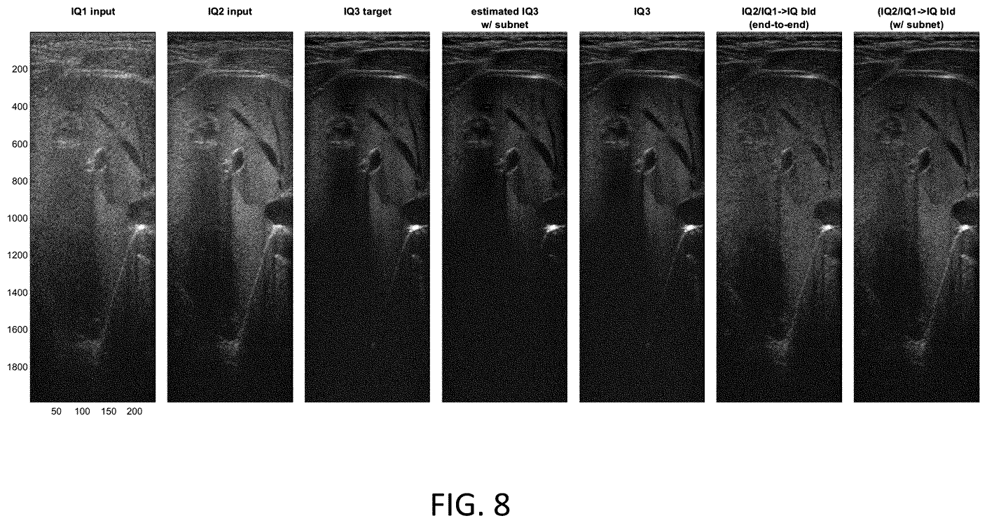

FIG. 8 illustrates example ultrasound images that are generated by the deep neural network based on a combined input of IQ1 data (fundamental frequency and third-order harmonics) and IQ2 data (second-order harmonics). Both the IQ1 data and the IQ2 data are obtained using an ultrasound imaging device 200. Example output images include an estimated IQ3 (third-order harmonics image), an IQ3 denoised (third-order harmonic) image. An IQ3 target image can be used for training the deep neural network in this example.

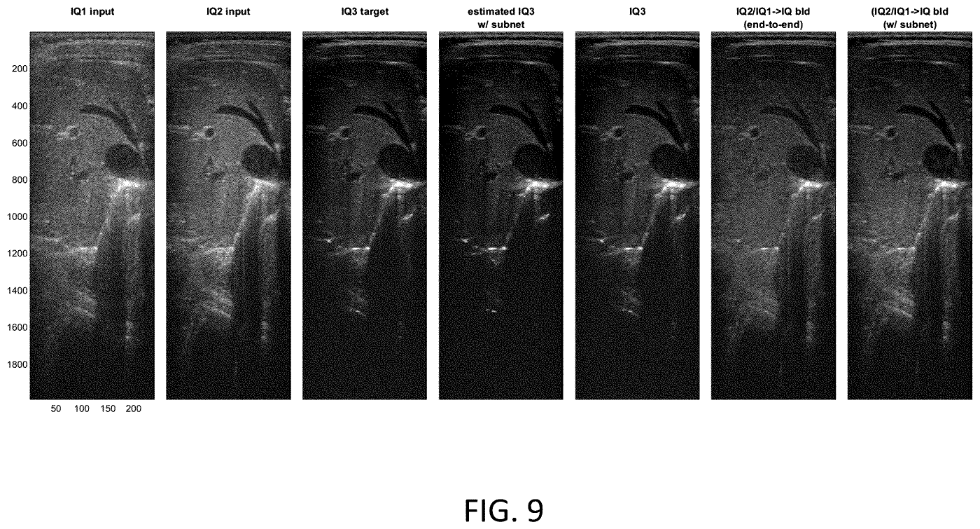

FIG. 9 illustrates another set of ultrasound images. Both the IQ1 data and the IQ2 data are obtained using an ultrasound imaging device 200. Example output images include an estimated IQ3 (third-order harmonic image), an IQ3 denoised (third-order harmonic) image. An IQ3 target image can be used for training the deep neural network in this example.

Embodiments of the deep learning network can include a deep-learning based framework having a fusion algorithm or fusion network that is trained to generate a fusion map based on input data that can be multiple different ultrasound images or multiple different IQ data. The deep-learning based framework can fuse feature-aware second- and third-order harmonics to obtain a desired image with improved resolution, fewer near-field artifacts, improved contrast, as well as deeper penetration. In one embodiment, one deep subnet can be trained using second-order harmonics and another deep subnet can be trained using third-order harmonics and the resulting images can be fused by the fusion network to obtain a desired target harmonic IQ data/image. The training of the fusion network can include training a depth-dependent fusion map and calculating a fusion loss based on the fusion map.

One approach to training a fusion map is an arrangement of feature extracting neural networks that feed into a prediction model. A fusion loss is calculated at the output of the prediction model and is propagated back to the feature extraction neural networks. The fusion map can be the fusion map of an internal layer of the feature extraction neural networks of a convolutional neural network.

In this disclosure, end-to-end (E2E) learning refers to training a complex machine learning system represented by a single model (typically a Deep Neural Network) that represents the complete machine learning system, instead of independently training the intermediate layers usually present in pipeline designs. In particular, the complete machine learning system can have two or more neural networks serving as components to a larger architecture. Training this architecture in an end-to-end manner means simultaneously training all components, i.e., training it as a single network.

In some embodiments, subnet training can be performed. In subnet training, one or more subnets are first trained for some arbitrary task in order to learn the task, e.g., feature extraction. Then the trained subnets are used in training a larger architecture with the subnets as components. Subnet training involves training of subnets then training the machine learning system in two separate phases.

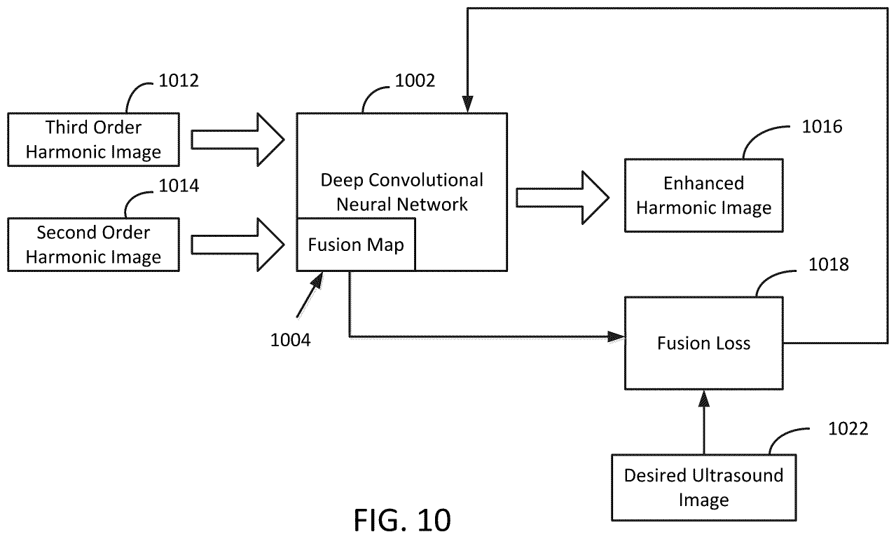

FIG. 10 is a block diagram of a deep neural network that is trained directly on a second-order harmonics image and a third-order harmonics image, in accordance with an exemplary aspect of the disclosure. A deep convolutional neural network model 1002 can be end-to-end trained with third-order harmonic images 1012 and second-order harmonic images 1014 to obtain an enhanced harmonic image 1016. A subnet 1004 of the deep convolutional neural network 1002 can be trained as a fusion map. A fusion loss function 1018 is calculated based on the enhanced harmonic image 1016 and target harmonic IQ data 1022, and the fusion loss is fed back to the deep convolutional neural network model 1002.

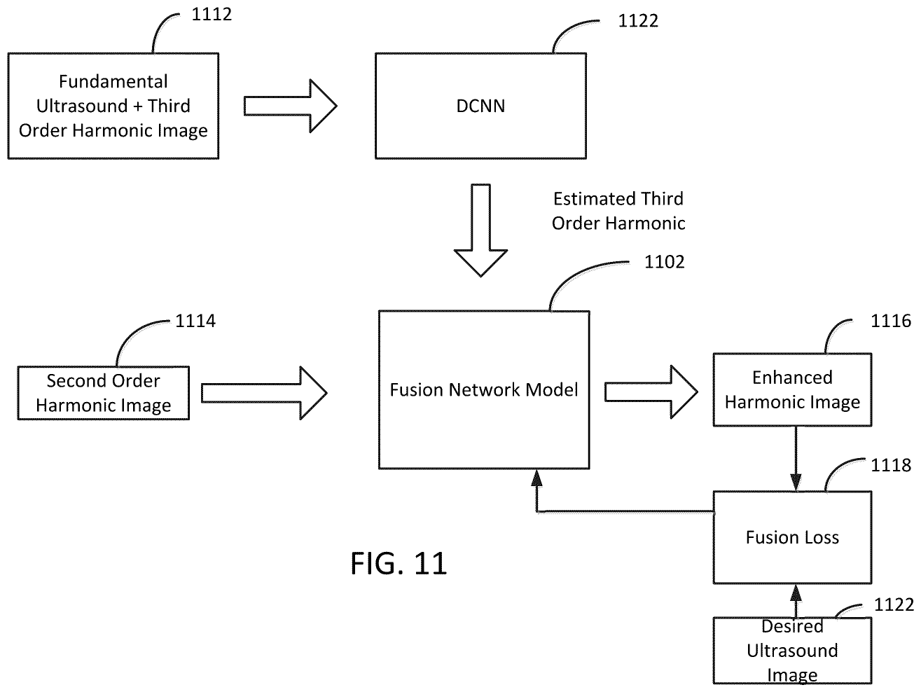

FIG. 11 is a block diagram of an alternative deep neural network that is trained to extract third-order harmonics and a fusion network model that is trained to fuse the extracted third-order harmonics with second-order harmonics, in accordance with an exemplary aspect of the disclosure. In some embodiments, a signal is obtained from the ultrasound transducer that is a combination of fundamental ultrasound frequency and the third order harmonic and simultaneously, a signal is obtained that is the second-order harmonic. A desired enhanced harmonic image 1116 can be determined using subnets. A deep convolutional neural network subnet 1122 can be trained to extract an estimated third-order image from an image of a fundamental ultrasound frequency and a third-order harmonic image 1112. A fusion network model subnet 1102 can be trained with the estimated third order harmonic images 1112 and second-order harmonic images 1114 to obtain an enhanced harmonic image 1116. A fusion loss function 1118 is calculated based on the enhanced harmonic image 1116 and the target harmonic IQ data 1122, and the fusion loss is fed back to the fusion network model 1102.

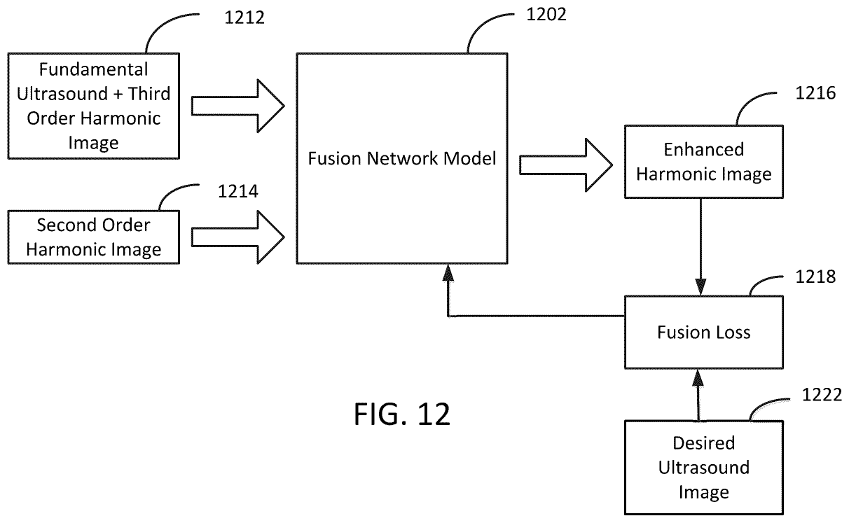

FIG. 12 is a block diagram of an alternative deep neural network that is trained directly on an IQ1 data/image and second-order harmonics, in accordance with an exemplary aspect of the disclosure. In some embodiments, a signal is obtained from the ultrasound transducer that is a combination of fundamental ultrasound frequency and the third-order harmonic and simultaneously, a signal is obtained that is the second-order harmonic. A fusion network model 1202 may be end-to-end trained with images of fundamental ultrasound frequencies and third-order harmonics 1212 and second-order harmonic images 1214 to obtain an enhanced harmonic image 1216. A fusion loss function 1218 is calculated based on the enhanced harmonic image 1216 and the target harmonic IQ data 1222, and the fusion loss is fed back to the fusion network model 1202.

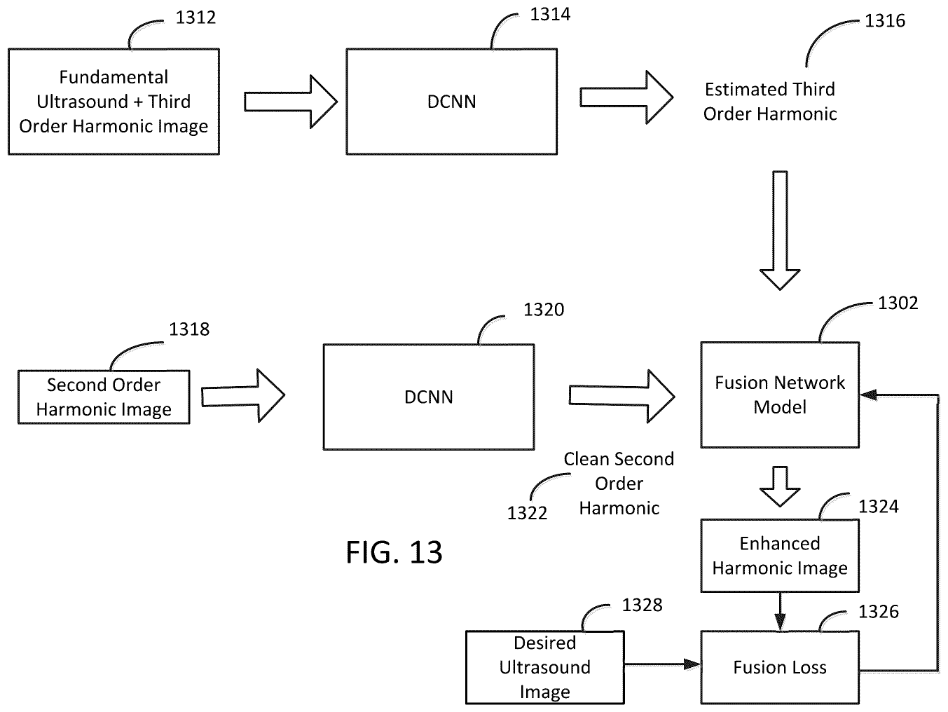

FIG. 13 is a block diagram of an alternative fusion network that is trained based on extracted third-order harmonics and cleaned second-order harmonics, in accordance with an exemplary aspect of the disclosure. An enhanced harmonic image 1324 can be determined using subnets. A first deep convolutional neural network subnet 1314 can be trained to extract an estimated third-order image 1316 from an image of a fundamental ultrasound frequency and a third-order harmonic image 1312. A second deep convolutional neural network subnet 1320 can be trained to denoise a second-order harmonic image 1318 to obtain a clean second-order harmonic image 1322. A fusion network model subnet 1302 may be trained with the estimated third-order harmonic images 1316 and the clean second-order harmonic images 1322 to obtain an enhanced harmonic image 1324. A fusion loss function 1326 is calculated based on the enhanced harmonic image 1324 and target harmonic IQ data 1328, and the fusion loss is fed back to the fusion network model 1302. In one embodiment, the fusion loss can be fed back to the first deep convolutional neural network 1314 and to the second deep convolutional neural network 1320.

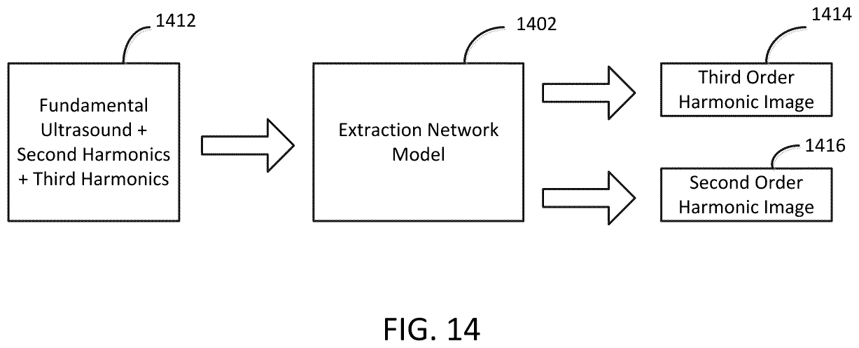

FIG. 14 is a block diagram of an extraction network that is trained to split second-order harmonics and third-order harmonics from IQ0 data/image, in accordance with an exemplary aspect of the disclosure. In an embodiment, fundamental frequency, second-order harmonics, and third-order harmonics are directly input to the deep neural network 1402 which is trained end-to-end to split the IQ0 data or ultrasonic image into IQ2 (second-order harmonic image 1416) and/or IQ3 (third-order harmonic image 1414). In one embodiment, the split second-order harmonic image 1416 and third-order harmonic image 1414 can then be input to a convolutional neural network, such as the deep convolutional neural network 1002 of FIG. 10, to obtain an enhanced harmonic image 1016.

In some embodiments, if higher-order harmonics are available, the deep neural network may be trained to obtain higher-order harmonic IQ data or ultrasonic images.

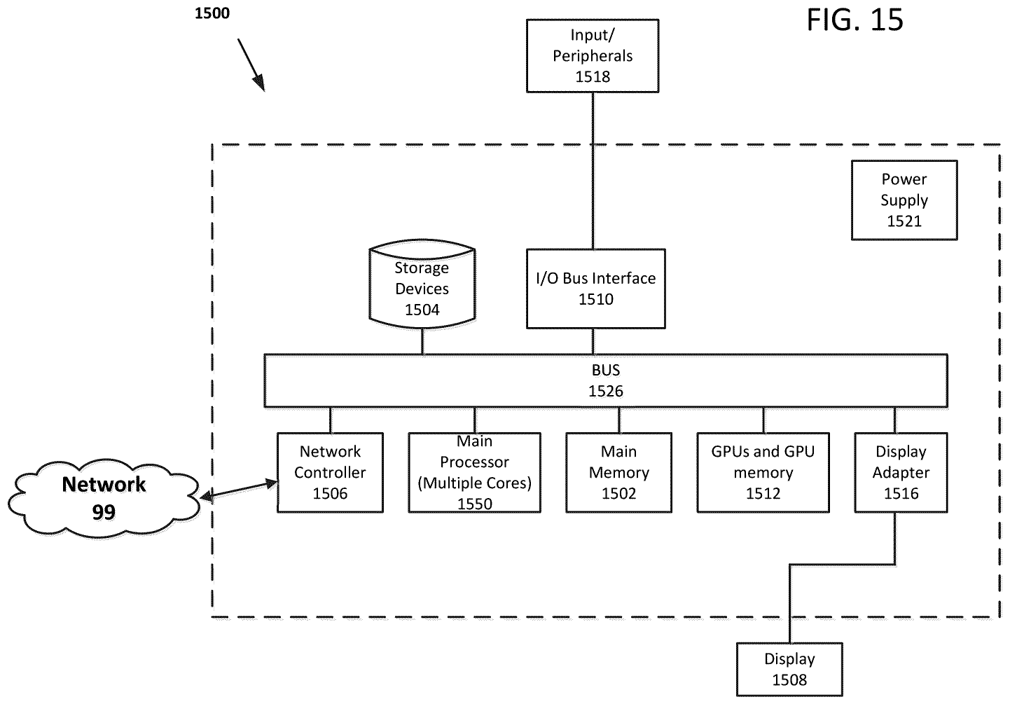

FIG. 15 is a block diagram illustrating an example computer system 204 for implementing the machine learning training and inference methods according to an exemplary aspect of the disclosure. The computer system may be an AI workstation running a server operating system, for example Ubuntu Linux OS, Windows Server, a version of Unix OS, or Mac OS Server. The computer system 1500 may include one or more central processing units (CPU) 1550 having multiple cores. The computer system 1500 may include a graphics board 1512 having multiple GPUs, each GPU having GPU memory. The graphics board 1512 may perform many of the mathematical operations of the disclosed machine learning methods. In other embodiments, the computer system 1500 may include a machine learning engine 1512. The machine learning engine 1512 may perform many of the mathematical operations of the disclosed machine learning methods. The computer system 1500 includes main memory 1502, typically random access memory RAM, which contains the software being executed by the processing cores 1550 and GPUs 1512, as well as a non-volatile storage device 1504 for storing data and the software programs. Several interfaces for interacting with the computer system 1500 may be provided, including an I/O Bus Interface 1510, Input/Peripherals 1518 such as a keyboard, touch pad, mouse, Display Adapter 1516 and one or more Displays 1508 (206), and a Network Controller 1506 to enable wired or wireless communication through a network 99. The interfaces, memory and processors may communicate over the system bus 1526. The computer system 1500 includes a power supply 1521, which may be a redundant power supply.

In some embodiments, the computer system 1500 may include a server CPU and a graphics card by NVIDIA, in which the GPUs have multiple CUDA cores.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. An apparatus, comprising:
processing circuitry configured to
receive first ultrasound data including at least one harmonic component;
apply the first ultrasound data to inputs of a trained deep neural network model that outputs enhanced ultrasound image data, the deep neural network model having been trained with training data including input ultrasound data and corresponding target ultrasound data having predetermined target features; and
output the enhanced ultrasound image data.

2. The apparatus of claim 1, wherein the first ultrasound data includes a fundamental frequency component and third-order harmonic component.

3. The apparatus of claim 2, wherein the processing circuitry is further configured to:
receive second ultrasound data including a second-order harmonic component; and
apply the first and second ultrasound data to the inputs of the trained deep neural network model to generate the enhanced ultrasound image data, which includes a third-order harmonic image fused with a second-order harmonic image.

4. The apparatus of claim 2, wherein the trained deep neural network outputs a third-order harmonic image based on the input first ultrasound data, and
the processing circuitry is further configured to:
receive second ultrasound data including a second-order harmonic component; and
apply the second ultrasound data to another trained deep neural network model that outputs a de-noised second-order harmonic image, the another deep neural network having been trained with training data including input ultrasound data and corresponding de-noised ultrasound data.

5. The harmonic imaging deep neural network of claim 4, wherein the processing circuitry is further configured to fuse the third-order harmonic image and the de-noised second order harmonic image to generate a fused image.

6. The apparatus of claim 1, wherein the first ultrasound data includes a third-order harmonics component, and the processing circuitry is further configured to:
receive second ultrasound data including a second-order harmonic component; and
apply the first and second ultrasound data to the inputs of the trained deep neural network model to generate the enhanced ultrasound image data, which includes a third-order harmonic image fused with a second-order harmonic image.

7. The apparatus of claim 1, wherein the first ultrasound data includes a fundamental frequency component, a second-order harmonics component, and a third-order harmonics component; and
the processing circuitry is further configured to apply the first ultrasound data to the trained deep neural network to generate the enhanced ultrasound image data, wherein the trained deep neural network model is trained to extract third-order harmonics data and second-order harmonics data from the first ultrasound data, and generate the enhanced ultrasound image data.

8. The apparatus of claim 1, wherein the first ultrasound data include a high-order harmonics component greater than third-order, and
wherein the trained deep neural network model reduces noise and generates an estimated high-order image from the first ultrasound data.

9. The apparatus of claim 1, wherein the enhanced ultrasound image data is enhanced fort a predetermined depth.

10. The apparatus of claim 1, wherein the predetermined target features of the enhanced harmonic image relate to a particular range of body mass index.

11. The apparatus of claim 1, wherein the predetermined target features of the enhanced harmonic image relate to particular demographic information.

12. The apparatus of claim 1, wherein enhanced ultrasound image data is a B-mode ultrasound image.

13. A method, comprising:
receiving first ultrasound data including at least one harmonic component;
applying the first ultrasound data to inputs of a trained deep neural network model that outputs enhanced ultrasound image data, the deep neural network model having been trained with training data including input ultrasound data and corresponding target ultrasound data having predetermined target features; and
outputting the enhanced ultrasound image data.

14. The method of claim 13, wherein the first ultrasound data includes a fundamental frequency component and a third-order harmonic component, and the method further comprises:
receiving second ultrasound data including a second-order harmonic component; and
applying the first and second ultrasound data to the inputs of the trained deep neural network model to generate the enhanced ultrasound image data, which includes a third-order harmonics image fused with a second-order harmonics image.

15. The method of claim 13, wherein the first ultrasound data includes a fundamental frequency component and third-order harmonic component, and the trained deep neural network outputs a third-order harmonic image based on the input first ultrasound data, and
the method further comprises:
receiving second ultrasound data including a second-order harmonics component; and
applying the second ultrasound data to another trained deep neural network model that outputs a de-noised second-order harmonic image, the another deep neural network having been trained with training data including input ultrasound data and corresponding de-noised ultrasound data.

16. The method of claim 15, further comprising:
fusing the third-order harmonic image and the de-noised second order harmonic image to generate a fused image.

17. The method of claim 13, wherein the first ultrasound data is a third-order harmonics component, and the method further comprises:
receiving second ultrasound data including a second-order harmonics component; and
applying the first and second ultrasound data to the inputs of the trained deep neural network model to generate the enhanced ultrasound image data, which includes a third-order harmonics image component fused with a second-order harmonics image component.

18. The method of claim 13, wherein the first ultrasound data includes a fundamental frequency component, a second-order harmonics component, and a third-order harmonics component, and
the method further comprises applying the first ultrasound data to the trained deep neural network to generate the enhanced ultrasound image data, wherein the trained deep neural network model is trained to extract third-order harmonics data and second-order harmonics data from the first ultrasound data, and generate the enhanced ultrasound image data.

19. The method of claim 13, wherein the first ultrasound data includes a high-order harmonic component greater than third-order, and
the enhanced ultrasound image data is a de-noised high-order harmonic image.

20. A non-transitory computer-readable medium storing a program that, when executed by processing circuitry, causes the processing circuitry to perform a method, comprising:
receiving first ultrasound data including at least one harmonic component;
applying the first ultrasound data to inputs of a trained deep neural network model that outputs enhanced ultrasound image data, the deep neural network model having been trained with training data including input ultrasound data and corresponding target ultrasound data having predetermined target features; and
outputting the enhanced ultrasound image data.
